(19) United States
(12) Patent Application Publication
(10) Pub. No.: US 2010/0087833 A1
(43) Pub. Date: Apr. 8, 2010

(54) CARTRIDGE FOR AN INTRAOCULAR IMPLANT

(75) Inventors: David Enfrun, Le Petit Bornand Les Glieres (FR); Gilles Bos, Plan-les-Ouates (CH); Stephane Mendras, Saint-Jorioz (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: XCELENS S.A., Plan-Les-Ouates (CH)

(21) Appl. No.: 12/300,381

(22) PCT Filed: May 5, 2006
(86) PCT No.: PCT/FR06/01011

§ 371 Date: Feb. 2, 2009

(51) Int. Cl. A61F 9/00

(52) U.S. Cl. 606/107

(57) ABSTRACT

A cartridge for an intraocular implant includes a loading chamber and an injection chamber communicating in a proximal portion of the cartridge. The cartridge further includes a piston chamber including a zone for communication with the loading chamber in a distal portion of the cartridge. The piston chamber includes a piston with an actuation end arranged to penetrate the loading chamber and to urge an intraocular implant located in the loading chamber through the injection chamber. An intraocular implant injector can include such a cartridge.

The present invention relates to a cartridge for an intraocular implant.

In the context of cataract surgery, it is known to destroy the crystalline lens of a patient using ultrasound, and then to remove it by means of an incision made in the patient's eye. The crystalline lens is then replaced by a lens which is also referred to as an “intraocular implant”.

Generally, the intraocular implants used have an optical portion provided with two lugs forming a haptic portion used to centre the intraocular lens in the capsular sac which contained the crystalline lens before the latter was destroyed.

In order to introduce the intraocular implant into the capsular sac, it is known to place the implant in a so-called “folding” cartridge and to connect the latter to an injector produced in the form of a syringe.

Such operations require extreme precision and the very nature of the eye necessitates a high degree of asepsis.

The known solutions are complex to implement owing to their nature and in respect of compliance with the requirements associated with this type of operation. The work of the surgeon is complicated thereby, and the chain of sterility has certain weak points, especially in connection with sterile separation between the elements which are to be in contact with the patient and those which have a purely mechanical role.

The object of the invention is to improve the situation.

To that end, the application proposes a cartridge for an intraocular implant, of the type comprising a loading chamber and an injection chamber communicating in a proximal portion of the cartridge. This cartridge also comprises a piston chamber which comprises a zone for communication with the loading chamber in a distal portion of the cartridge and which receives a piston provided with an actuation end.

This actuation end is arranged to penetrate the loading chamber and to push an intraocular implant received in the loading chamber through the injection chamber.

Such a cartridge is particularly advantageous because it enables the number of manipulations during the surgical operation to be reduced. For, as the piston has already been received in the cartridge, it is no longer necessary to introduce it at the same time as the implant, which reduces possible errors of manipulation.

Furthermore, such a cartridge also improves sterile isolation because the pre-loaded piston enables the components which are in contact with the implant, on the one hand, and the components which are intended purely to permit the injection of the latter, on the other hand, to be separated.

Thus, the only operation that could jeopardize sterility is that of introducing the implant, and this operation is generally well controlled. In addition, since the surgeon no longer has to deal with the loading of the piston and the problems of sterility which accompany this loading, he is better able to concentrate on the rest of the operation.

In various embodiments, the cartridge may have the following features:
the loading chamber and the piston chamber are substantially cylindrical (in the broad sense of a cylindrical surface, of any directrix, for example a circle or an ellipse) and have (a) substantially identical internal radius (radii);
the loading chamber has a frustoconical shape converging towards the proximal portion of the cartridge;
in the region of the injection chamber, the loading chamber has a first diameter substantially equal to the diameter of the injection chamber, and, in the region of the piston chamber, a second diameter substantially equal to the diameter of the piston chamber;
the piston is pre-positioned in such a manner as to fill the piston chamber substantially completely and to isolate the cartridge in a sterile manner;
the injection chamber has a frustoconical shape diverging towards the loading chamber;
the injection chamber, the loading chamber and the piston chamber are produced from polypropylene;
the piston is produced from elastomeric material;
the loading chamber comprises wings suitable for co-operating in order to close the cartridge.

Other features and advantages of the invention will emerge more clearly on reading the following description of examples given by way of non-limiting illustration and stemming from the drawings, in which:

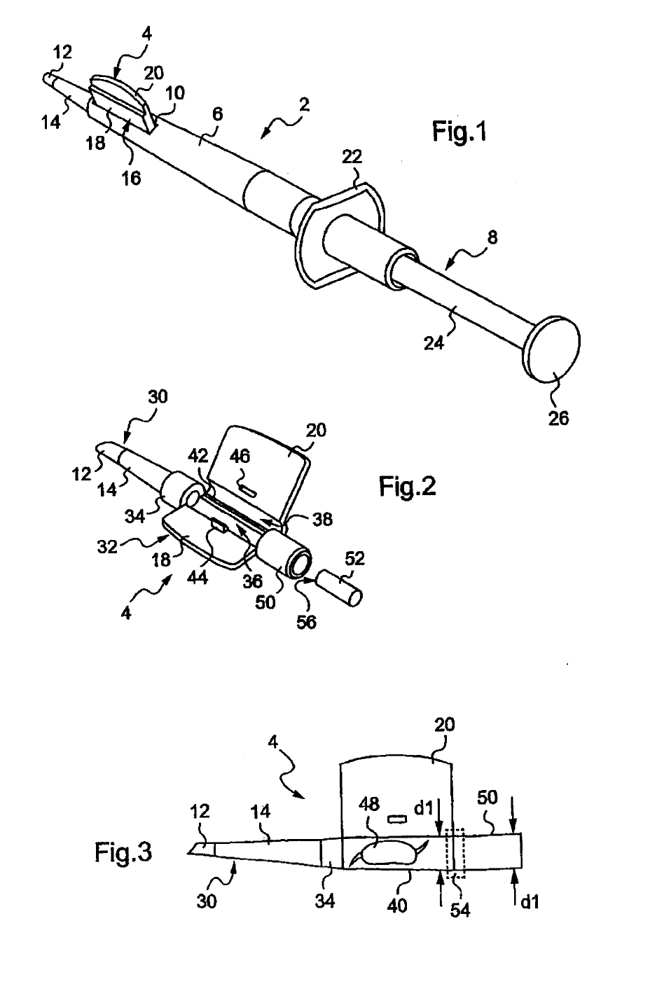

FIG. 1 is a perspective view of an intraocular implant injector comprising a cartridge according to the invention;

FIG. 2 is a perspective view of the cartridge of FIG. 1 before the intraocular implant is loaded;

FIG. 3 is a sectional view along the longitudinal axis of the cartridge of FIG. 2, after an intraocular implant has been loaded;

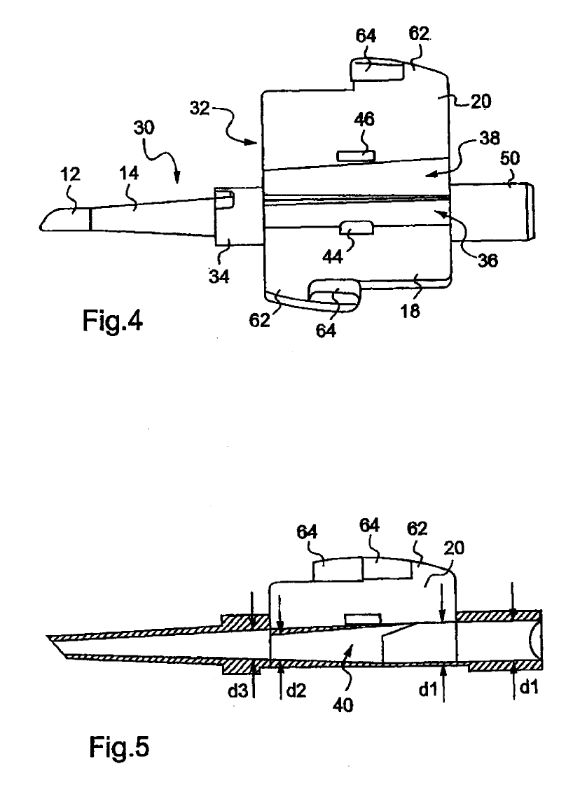

FIG. 4 is a perspective view of a variant of a cartridge; and

FIG. 5 is a sectional view along the longitudinal axis of the cartridge of FIG. 4.

The following drawings and description basically contain elements of a definitive nature. They can therefore be used not only better to explain the present invention but also to contribute to the definition thereof, where appropriate.

Hereinafter, the expression “loading an intraocular implant” means its arrangement in a folding cartridge and its folding therein. In addition, an intraocular implant is not arranged alone in the cartridge: it is loaded with its medium, which may be a solution facilitating sliding in the cartridge, for example of the viscoelastic type.

As can be seen in FIG. 1, an intraocular implant injector comprises a folding cartridge 4 accommodated in an injector body 6. The injector 2 is in the general form of a syringe, that is to say, the body 6 has a hollow cylindrical general shape, which receives a pushing member 8, and a pointed end.

The body 6 comprises at its proximal end a housing 10 which is dimensioned to receive the cartridge 4 securely. The cartridge 4 comprises an injection head 12 which is preceded by a canula 14.

The cartridge 4 is held in place in the housing 10 by a closing mechanism 16. The closing mechanism 16 may be formed, for example, by a recess in the housing 10, in which recess two wings 18 and 20 of the cartridge 4 are accommodated.

The closing mechanism 16 ensures that the wings 18 and 20 are locked in terms of translation and rotation. The wings 18 and 20 are held one against the other, and the cartridge 4 cannot come out of the housing 10. The wings 18 and 20 will be described in more detail by means of FIGS. 2 and 3.

The body 6 is separated between its proximal portion and its distal portion by a collar 22. The collar 22 extends substantially radially and acts as a finger-rest for the index finger and the middle finger of a hand.

The pushing member 8 is produced in the general form of a rod 24 which penetrates the body 6 and the proximal end of which is capable of penetrating a distal end of the cartridge 4. At its distal end, the rod 24 has a generally circular head 26 on which the thumb of a hand can press in order to cause the rod 24 to slide in the body 6.

In order to inject the intraocular implant into the patient's eye, the surgeon starts by folding the intraocular implant in the cartridge 4 as will be described hereinafter. He then secures the cartridge 4 in the housing 10 of the body 6.

Finally, the surgeon presses on the head 26 with his thumb while holding the finger-rest 22 with his middle finger and his index finger and causes the rod 24 to slide until the proximal end of the rod 24 penetrates the cartridge 4 to inject the intraocular implant.

In the example described here, the body 6 and the pushing member 8 are produced by moulding from plastics material of any type, and the cartridge 4 is produced by moulding from polypropylene. Preferably, the polypropylene used to mould the cartridge 4 does not have any additional elements and is of medical grade, but other types of polypropylene may be suitable.

The person skilled in the art will appreciate that a large variety of injectors exists. He will understand, in particular, that the only requirements with respect to the injector relate to compatibility in terms of receiving the cartridge and holding it in position, and the presence of a pushing member having an end enabling the pre-positioned piston to be pushed into the cartridge.

The cartridge 4 can assume two extreme positions:
an opened-out position, outside the body 6, represented in FIG. 2, in which the cartridge 4 is capable of receiving an intraocular implant for the loading (folding) thereof;
a closed position, shown in longitudinal section in FIG. 3, in which the cartridge 4 contains an intraocular implant and is capable of being mounted on the injector 2 in order to inject the implant.

As can be seen in FIG. 2, the cartridge 4 comprises an injection chamber 30 in its proximal portion. The injection chamber 30 is formed by the injection head 12 and the canula 14. The injection head 12 has a generally frustoconical shape converging towards the proximal end of the cartridge 4 and intersected by a plane, thus forming an end bevelled at approximately 45°.

The end of the cartridge 4 thus has a profile which is particularly suitable for injecting the implant in the manner of the head of a syringe. Depending on the application, the angle of the bevel formed by this end may vary.

The canula 14 has a generally frustoconical outer shape converging towards the proximal end of the cartridge 4. Along its longitudinal axis, the canula 14 has a frustoconical bore which enables the intraocular implant to be guided when it is injected. In addition, this bore enables the implant to be progressively rolled up, which reduces its size in the region of the injection head 16, and consequently the size of the incision necessary to carry out the injection.

In its distal portion, the injection chamber 30 is connected to a loading chamber 32 which is shown opened out in FIG. 2. The chamber 32 comprises a communication end 34 and two open ducts 36 and 38. The ducts 36 and 38 have complementary shapes so as to form a closed loading duct 40 (see FIG. 3) when the cartridge 4 is closed.

The ducts 36 and 38 are extended by the wing 18 and the wing 20, respectively, which enable the cartridge 4 to be opened out and closed by being moved towards or away from each other. In the embodiment described, the duct 38 and its wing 20 are mounted to pivot relative to the duct 36 and its wing 18 by means of a hinge 42. The cartridge 4 is kept in its closed position by the closing mechanism 16 of the body 6.

In order to prevent the intraocular implant from becoming jammed between the wings 18 and 20 when the cartridge 4 is closed, the wing 18 has a projecting portion 44 and the wing 20 has a housing 46 homologous to the projecting portion 44.

The co-operation of the projecting portion 44 and the housing 46 also enables the wings 18 and 20 to be centred relative to each other when the cartridge 4 is closed.

The centring thus effected permits better folding of the intraocular implant and correct symmetry for the duct 40.

The implant is folded by positioning it in the chamber 32 when the latter is in the opened-out position and by closing the chamber 32 by moving the wings 18 and 20 against each other until the projecting portion 44 is accommodated in the housing 46.

Numerous variants will occur to the person skilled in the art for producing the ducts 36 and 38 in a manner pivoting with respect to each other by means of the wings 18 and 20.

The interior shape of the ducts 36 and 38 determines the manner in which the intraocular implant will be folded when the cartridge 4 is closed, and also the later injection of the intraocular implant through the injection chamber.

The intraocular implant is folded by being rolled up on itself owing to the shape which is given to the ducts 36 and 38. It then has the general shape of the implant 48 shown in FIG. 3.

The outer casing of the duct 40 formed by joining the ducts 36 and 38 is generally cylindrical, and the wings 18 and 20 have a generally rectangular shape, the wing 18 being smaller than the wing 20 in order to facilitate the opening of the cartridge 4. Here again, numerous variants will occur to the person skilled in the art for opening and closing the cartridge 4.

In the distal portion of the cartridge 4, the loading chamber 32 is connected to a piston chamber 50 which receives a piston 52. As can be seen in FIG. 3, the piston chamber 50 has a communication zone 54 which communicates with the loading chamber 32.

As mentioned above, the closed duct 40 has a shape determined by that of the ducts 36 and 38. This shape is fairly similar to that of a cylinder without necessarily being exactly cylindrical, and the inside diameter d1 of the duct 40 is equal to approximately 3 mm. The piston chamber 50 has a generally cylindrical shape hollowed out to form a bore having a diameter equal to that of the duct 40.

The piston 52 is manufactured from an elastomer and may be produced in a general manner from any elastomeric material of suitable deformability. The piston 52 has a generally cylindrical shape, with a diameter slightly larger than the inside diameter of the piston chamber 50, so that the piston 52 fills the latter in a sealed manner.

When the surgeon presses on the pushing member 8 of the injector 2, the end of the latter pushes the piston 52 by the rear thereof through the piston chamber 50. The piston 52 progressively passes a communication zone 54 and then penetrates the loading chamber 32 until an actuation end 56 of the piston 52 pushes the intraocular implant 48 and the medium surrounding it through the injection chamber 30 and into the capsular sac of the patient's eye.

The piston chamber 50 and the piston 52 of the invention therefore permit sterile isolation of the cartridge 4 with the rest of the injector 2. Thus, the surgeon no longer has to deal with this aspect and he can focus on the operation itself.

Moreover, the integration of the piston chamber 50 with the piston 52 ensures that the intraocular implant 48 cannot become jammed between the duct 40 and the piston 52 during the loading thereof. The form of the piston 52 also ensures that the intraocular implant 48 and its medium are pushed in an optimum and facilitated manner.

Finally, since the cartridge 4 is produced from polypropylene, the thickness of the piston chamber 50 protects the piston 52 from the ambient light radiation which could impair its deformation properties. The cartridge 4 can therefore be stored for a relatively long time without the piston 52 being impaired to the point of becoming unusable.

FIG. 4 shows a variant of a cartridge which has other advantages. As can be seen in this Figure, the loading chamber 32 is produced in a different manner.

Thus, the ducts 36 and 38 have been modified so that, once the cartridge 4 has been closed, the duct 40 is no longer substantially cylindrical but frustoconical, converging towards the proximal portion of the cartridge 4. The frustoconical shape of the duct 40 enables the intraocular implant to be guided and rolled up as described above.

As can be seen in FIG. 5, the duct 40 thus has a diameter d1 equal to that of the piston chamber 50 in the region of the latter and a diameter d2 substantially equal to 2.5 mm in the region of the injection chamber 30.

In the example shown, the diameter d2 is slightly smaller than the diameter d3 of the injection chamber 30 in the region in which the loading chamber 32 communicates with the injection chamber 30. The diameter d2 may be equal to the diameter d3.

In addition, the wings 18 and 20 are produced in a different manner in this variant. The wings 18 and 20 still have a generally rectangular shape but they are produced to be identical in this embodiment and are arranged as mirror images of each other.

The wings 18 and 20 each comprise, in their most eccentric region relative to the duct 40, a catch portion 64 which extends between one of the edges of the rectangle and the middle of the latter.

In the region of its end located in the middle of the rectangle, each catch portion 64 is provided with a lug 62 which extends towards the wing lying opposite it. Thus, when the wings 18 and 20 are folded up against each other, the lugs 62 of each wing co-operate in a resilient manner to form a “jack-knife” closure and to ensure that the cartridge 4 is kept in its closed position.

The above description of the embodiment is given in order to permit a better understanding of the invention. Nevertheless, the scope of the invention does not have to be limited to this embodiment and it encompasses all of the variants which the person skilled in the art can envisage and is defined by the appended claims.

1-10. (canceled)

11. A cartridge for an intraocular implant, comprising:
a loading chamber;
an injection chamber communicating in a proximal portion of the cartridge; and
a piston chamber including a zone for communication with the loading chamber in a distal portion of the cartridge, the piston chamber receiving a piston with an actuation end, which actuation end is configured to penetrate the loading chamber and to push an intraocular implant received in the loading chamber through the injection chamber.

12. A cartridge according to claim 11, wherein the loading chamber and the piston chamber are substantially cylindrical and have a substantially identical internal radius.

13. A cartridge according to claim 11, wherein the loading chamber has a frustoconical shape converging towards the proximal portion of the cartridge.

14. A cartridge according to claim 13, wherein, in a region of the injection chamber, the loading chamber has a first diameter substantially equal to the diameter of the injection chamber, and, in a region of the piston chamber, a second diameter substantially equal to the diameter of the piston chamber.

15. A cartridge according to claim 11, wherein the piston is pre-positioned to fill the piston chamber substantially completely and to isolate the cartridge in a sterile manner.

16. A cartridge according to claim 11, wherein the injection chamber has a frustoconical shape diverging towards the loading chamber.

17. A cartridge according to claim 11, wherein the injection chamber, the loading chamber, and the piston chamber are produced from polypropylene.

18. A cartridge according to claim 11, wherein the piston is produced from elastomeric material.

19. A cartridge according to claim 11, wherein the loading chamber comprises wings configured to cooperate to close the cartridge.

20. An injector for an intraocular implant, comprising:
a hollow body receiving a pushing member, and receiving at a proximal end a cartridge according to claim 11,
wherein the pushing member comprises a pushing end arranged to push the piston received in the piston chamber through the loading chamber.
